(19) United States
(12) Patent Application Publication
(10) Pub. No.: US 2018/0275086 A1
(43) Pub. Date: Sep. 27, 2018

(54) METHOD FOR MODIFYING A WORKING ELECTRODE FOR A SENSOR DETECTING A METAL DISSOLVED IN TRACE AMOUNTS IN A LIQUID, ELECTRODE, SENSOR AND USE OF SAME

(71) Applicants: Universite De Pau Et Des Pays De L'Adour, Pau (FR); Centre National De La Recherche Scientifique, Paris (FR)

(72) Inventors: Corinne Parat, Navailles Angos (FR); Isabelle Le Hecho-Bouroullec, Gelos (FR)

(21) Appl. No.: 15/763,924

(22) PCT Filed: Sep. 27, 2016
(86) PCT No.: PCT/EP2016/073004

§ 371 Date: Mar. 28, 2018

(30) Foreign Application Priority Data
Sep. 28, 2015 (FR) 15 59131

(51) Int. Cl. G01N 27/30; G01N 27/42; G01N 33/18

(52) U.S. Cl. CPC G01N 27/308; G01N 33/1813; G01N 27/42

(57) ABSTRACT

Provided is a method for modification of a working electrode for a sensor which is able to detect at least one dissolved trace metallic element in a liquid, such an electrode, which is obtained by this method, a sensor incorporating it and the use thereof for detecting and possibly quantifying a metallic element in liquids. This method includes a step of depositing a polymeric film based on a poly(catechol) on the surface by electropolymerisation of pyrocatechol in a first environment with a pH between 6 and 8, the method subsequently includes conditioning the working electrode having a succession of reduction-oxidation cycles in a second environment with a pH of at least 10, in order for the modified working electrode to be electroactive in a field of electroactivity, measured by cyclic voltammetry, ranging from −1 V to +1 V while being able to detect copper and/or lead as the metallic element.

The present invention relates to a method for the modification of a working electrode for a sensor which is able to detect at least one dissolved trace metallic element in a liquid, such an electrode, which is for example screen-printed and obtained by this method, a sensor incorporating it and the use thereof for detecting and possibly quantifying said at least one metallic element in liquids. The invention applies in a general manner to the monitoring and control, before or after treatment, of various types of water likely to be contaminated with or polluted by metals or metalloids present in the dissolved state in the form of traces (i.e. in the form of a dispersion of elements selected from chromium, nickel, tin, lead, copper, silver, mercury, uranium, selenium, arsenic, antimony and cobalt, without limitation), or of industrial baths whose composition is to be analysed.

It is known that industrial activities can lead to increased concentrations of metals in industrial water, waste water or other water until the legal toxicity limits are reached. It is therefore necessary for manufacturers to carry out controls on or to monitor these waters, in particular as they leave the factory, by means of regular analyses of the discharge in order to minimise the pollution of the receiving environments. For this purpose, samples must be taken on-site and then transferred to the laboratory for analysis, which has the disadvantage of involving a delay that can be relatively long between each sampling and obtaining the analysis results.

In recent years, attempts have therefore been made to design sensors that can be integrated into a monitoring station for such industrial waters or baths, with the recent development of sensors with a working electrode based on carbon modified with a mercury film. In order to stop contributing to the further pollution of the environment through the harmful metal that is mercury, recent attempts have been made to develop new working electrodes for the detection of trace metallic elements which fully satisfy the criteria of sensitivity at the μg/L range, of reproducibility (i.e. repeatability) of the measurement signals, of stability over time and of robustness with respect to the specific conditions of the solution considered containing the metal to be detected which excludes mercury.

For this purpose, working electrodes modified with a bismuth, gold, copper or silver film have been proposed, but with a sensitivity and reproducibility defect which depends on the pH of the solution for a working electrode modified with bismuth, with a high underflow which is variable depending on the oxygen for a working electrode modified with gold and a limit of detection that is too high for a working electrode modified with copper or silver.

The article Davis, J., Vaughan, D. H., Cardosi, M. F., 1998, Modification of catechol polymer redox properties during electropolymerization in the presence of aliphatic amines, Electrochimica Acta 43(3-4), 291-300, discusses deposition by electropolymerisation of pyrocatechol on a carbon material-based working electrode (made from glassy carbon), using the technique of cyclic voltammetry in a phosphate buffer solution with a neutral pH containing pyrocatechol supplemented with ethylene diamine. However, this article does not relate to the use of this working electrode modified in this way to detect trace metallic elements.

The article Khoo, S. B., Zhu, J., 1999, Poly(catechol) Film Modified Glassy Carbon Electrode for Ultratrace Determination of Cerium (Ill) by Differential Pulse Anodic Stripping Voltammetry, Electroanalysis 11(8), 546-552, discusses deposition by electropolymerisation on such a glassy carbon-based working electrode of a film made from a poly(catechol) exclusively for the detection of cerium Ill. This deposition is carried out in a basic environment, by cyclic voltammetry in a soda solution at 0.1 mol/L containing pyrocatechol, and is followed by scans in another soda solution to remove undesirable monomers within the film.

A major disadvantage of the method for the modification of a working electrode according to this latter article is that it is limited to the detection of cerium, therefore not being suitable for the detection of other trace metals such as copper or lead with satisfactory sensitivity and stability in particular.

One object of the present invention is to propose a novel method for the modification of a carbon material surface of a working electrode which overcomes the aforementioned disadvantages by being able to detect, within a sensor, at least one dissolved trace metallic element in a liquid, which has a standard oxidation-reduction potential which is very different from that of cerium.

This object is achieved in that the Applicant has unexpectedly discovered that a deposition by electropolymerisation in a first environment with a pH of between 6 and 8 of pyrocatechol to form a film based on a poly(catechol) on a carbon material surface of a working electrode, followed by conditioning of the working electrode by reduction-oxidation cycles in a second environment with a pH of at least 10, makes it possible to make the working electrode electroactive in a field of electroactivity measured by cyclic voltammetry ranging from −1 V to +1 V, making it particularly suitable for detecting, within a sensor incorporating this electrode, copper and/or lead as dissolved trace metallic element(s) in a liquid with improved sensitivity, reproducibility, stability and robustness compared to existing sensors.

In other words, a method for the modification according to the invention of a carbon material surface of such a working electrode comprises a step a) of depositing a polymeric film based on a poly(catechol) on said surface by electropolymerisation of pyrocatechol in a first environment with a pH of between 6 and 8, and this method is such that it subsequently comprises a step b) of conditioning said working electrode comprising a succession of reduction-oxidation cycles in a second environment with a pH equal to or greater than 10, in order for said modified working electrode to be electroactive in this field of electroactivity ranging from −1 V to +1 V while being able to detect copper and/or lead as said at least one metallic element.

It should be noted that this detection of trace metallic elements whose oxidation/reduction couples have a standard oxidation-reduction potential of between −1 V and +1 V and which include, in particular, copper and lead, was by no means predictable in view of the aforementioned article by Khoo, S. B., Zhu, J., and provides evidence, on the contrary, of a technical prejudice being overcome in view of this article since it taught that the electropolymerisation of pyrocatechol in a basic environment on a similar working electrode could only be used for the detection of trace cerium, whose standard oxidation-reduction potential Ce⁴⁺/C³⁺ close to 1.72 V is located well outside the range of the invention of between −1 V and +1 V.

It should also be noted that the Applicant has discovered that step b) of conditioning according to the invention makes it possible to make the poly(catechol) film stable in use over time and to provide it with improved detection sensitivity at the same time, compared to the aforementioned films of the prior art deposited on working electrodes.

It should be further noted that the Applicant has verified that no detection signal of a metallic element such as copper is obtained if this step b) of conditioning in a very basic environment is not implemented.

The expression “based on”, used to define the composition of the polymeric film deposited on the working electrode, means, in the present description, that this film comprises poly(catechol) predominantly in mass (i.e. with a mass fraction greater than 50%, or even greater than 75%), it having been specified that this film according to the invention could comprise other monomeric or polymeric compounds as a minority in mass such as polyhydroxybenzene derivative-type monomers (not limited to pyrocatechol) and/or other additives such as, for example, ethylene diamine, as explained below.

“Cyclic voltammetry” (abbreviated to CV) means, in a known manner, a technique based on the measurement of the current which is obtained by a linear scan with a potential between the defined potentials and which results from oxidation and reduction cycles of the elements present in a solution.

According to another feature of the invention, step b) may be implemented in a soda solution which is free from said at least one metallic element and which preferably has a pH of between 11 and 13.5 and for example a concentration of 0.1 mol/L.

Advantageously, step b) may be implemented by stripping chronopotentiometry, carrying out between 5 and 15 of said reduction-oxidation cycles.

“Stripping chronopotentiometry” also means, in a manner known in the present description, an electrochemical method which essentially comprises electrodeposition on the electrode by means of a sufficiently reducing potential applied for a given period of time with stirring, and stripping by means of an oxidation current applied in such a way that the reduced metals are re-oxidised.

Even more advantageously, step b) may be implemented for each of said cycles with the application of a reducing and then oxidising potential for a period of between 30 seconds and 90 seconds, preferably of between 50 and 70 seconds.

As a variant of stripping chronopotentiometry, it is possible to use electrochemical techniques using anodic stripping voltammetry (ASV) such as SWASV (square wave anodic stripping voltammetry) or DPASV (differential pulse anodic stripping voltammetry).

This method for the modification of the working electrode according to the invention preferably further comprises, after step b), a step c) of stabilising said working electrode comprising a repetition of other reduction-oxidation cycles in a third environment which has a pH of between 4 and 5 and which comprises said at least one metallic element to be detected, to stabilise said polymeric film.

It should be noted that this step c) is required in order to optimise the stability of the detection signal emitted by the sensor for a metallic element such as copper and/or lead in particular, and therefore in order for the sensor incorporating the working electrode modified in this way to be able to be used in good conditions.

Even more preferably, said third environment is an acetate buffer solution preferably at 0.1 mol/L which comprises copper and/or lead as said at least one metallic element at a concentration of between 50 ng/L and 100 μg/L.

Also preferentially, step c) is implemented by stripping chronopotentiometry, carrying out at least 25 of said other cycles.

Advantageously, said first environment in which step a) is implemented may be a phosphate buffer solution preferably at 0.1 mol/L which has a pH of between 6.5 and 7.5 and which comprises said pyrocatechol at a concentration of between 5 mmol/L and 15 mmol/L.

Even more advantageously, step a) is implemented by cyclic voltammetry, with a plurality of scans ranging from −0.8 V to +0.8 V carried out at a speed of at least 200 mV/s.

According to another feature of the invention, step a) may further comprise an initial addition of ethylene diamine to said first environment, at a concentration of between 5 mmol/L and 15 mmol/L.

It should be noted that the Applicant has demonstrated that the ethylene diamine initially added to the pyrocatechol solution in particular makes it possible to improve the stability of the film over time (this ethylene diamine acts as a catalyst for the electropolymerisation reaction).

A working electrode according to the invention is able to detect, within said sensor, at least one dissolved trace metallic element in a liquid that can advantageously have a pH of between 2 and 8, the working electrode having a surface based on a carbon material coated with a polymeric film based on a poly(catechol), and said film defining a working surface of the working electrode.

This working electrode is such that its working surface comprises the product of reduction-oxidation reactions by stripping chronopotentiometry of the poly(catechol) with a soda solution which functionalises it, the working electrode being electroactive in a field of electroactivity, measured by cyclic voltammetry, ranging from −1 V to +1 V while being able to detect copper and lead as said at least one metallic element.

It should be noted that this functionalisation of the poly(catechol) by the soda solution added during the aforementioned step b) makes this film according to the invention based on the functionalised poly(catechol) structurally different from a film based on a poly(catechol) which has not been functionalised by this reaction, the product of which thus defines a working electrode according to the invention.

According to another feature of the invention, this working electrode according to the invention is furthermore such that said working surface also comprises the product of reduction-oxidation reactions by stripping chronopotentiometry of said poly(catechol) with an acetate buffer solution which comprises copper and/or lead as said at least one metallic element at a concentration of between 50 ng/L and 100 μg/L, the copper and/or lead having reacted with said film.

It should be noted that this reaction product of the copper and/or lead with the film is reflected in the working electrode by the presence of one or both of these metals in this film, which thereby has a structure which is different from that of a film which has not been subject to step c) mentioned above.

A sensor according to the invention is able to detect at least one dissolved trace metallic element in a liquid, the sensor comprising a working electrode, a counter electrode and a reference electrode, and it is characterised in that said working electrode is as defined above, the sensor being able to detect said metallic elements which have a standard oxidation-reduction potential of between −1 V and +1 V and which include copper and lead.

It should be noted that metallic elements (i.e. metals or metalloids) other than Cu and Pb are able to be detected and quantified by such a sensor according to the invention, such as Cr, Ni, Sn, Ag, Hg, U, Se, As, Sb and Co, without limitation.

It should also be noted that an unmodified working electrode of the prior art (i.e. based on a carbon material such as carbon black or graphite) was only able to measure metals in solution having a standard oxidation-reduction potential of between −0.4 V and +1.2 V.

According to another feature of the invention, the sensor may have a sensitivity, expressed as the limit of detection of said at least one metallic element, which is less than 1.5 μg/L in particular for copper and lead and may even be less than 70 ng/L in the case of lead, for example.

It should be noted that the limit of detection (abbreviated to LOD) corresponds in a known manner to the moment when a signal is visible but cannot be integrated:

LOD=[b+(3·σ_{b})]/a with:

b: intercept of the equation of the trend curve (y=ax+b),

a: slope of the equation of the trend curve, and

σ_{b}: standard deviation of the intercept b.

it should also be noted that these limits of detection are as good as, if not better than, those which are usually obtained with a working electrode modified with a mercury film.

According to the invention, said sensor may be used to detect and possibly quantify in situ, for example in a monitoring station (following the example of a pH electrode), said at least one metallic element in solution in industrial waters or baths, before and/or after their treatment, which are selected, for example, from surface waters, groundwater, industrial discharge, water from sewage treatment plants, drainage and runoff water, water from polluted and unpolluted soils and leachate from landfills.

It should be noted that this sensor according to the invention has the following advantages in particular:
the absence of mercury in its working electrode makes it possible to market the sensor without any restrictions and to carry out environmental monitoring in situ without any risk of contaminating the environment concerned;
the relatively long lifespan of the film modifying the working electrode according to the invention enables the sensor to be used for several days or even several weeks (which was not the case for sensors with working electrodes modified with a mercury film) and without requiring any maintenance action;
the stability and robustness of the film fitted in the sensor enable it to be used without particular precautions in monitoring stations or complex environments, such as charged, industrial-type solutions;
the sensitivity of the sensor is compatible with use in particularly natural environments where the concentrations of trace metallic elements are in the μg/L range;
this sensor can be used at high frequency without taking samples and thus makes it possible to reduce the analysis costs and eliminate the risk of contamination of the sample between the environment concerned and the laboratory;
it is possible to modify the shape and the size of the working electrode of the sensor as well as the thickness of the film covering it by modifying the number of electropolymerisation cycles, depending on the desired concentrations of the metallic element(s); and
this sensor has reduced manufacturing costs.

Other features, advantages and details of the present invention will emerge when reading the following description of several exemplary embodiments of the invention given in an illustrative and non-limiting manner, the description being completed with reference to the accompanying drawings, in which:

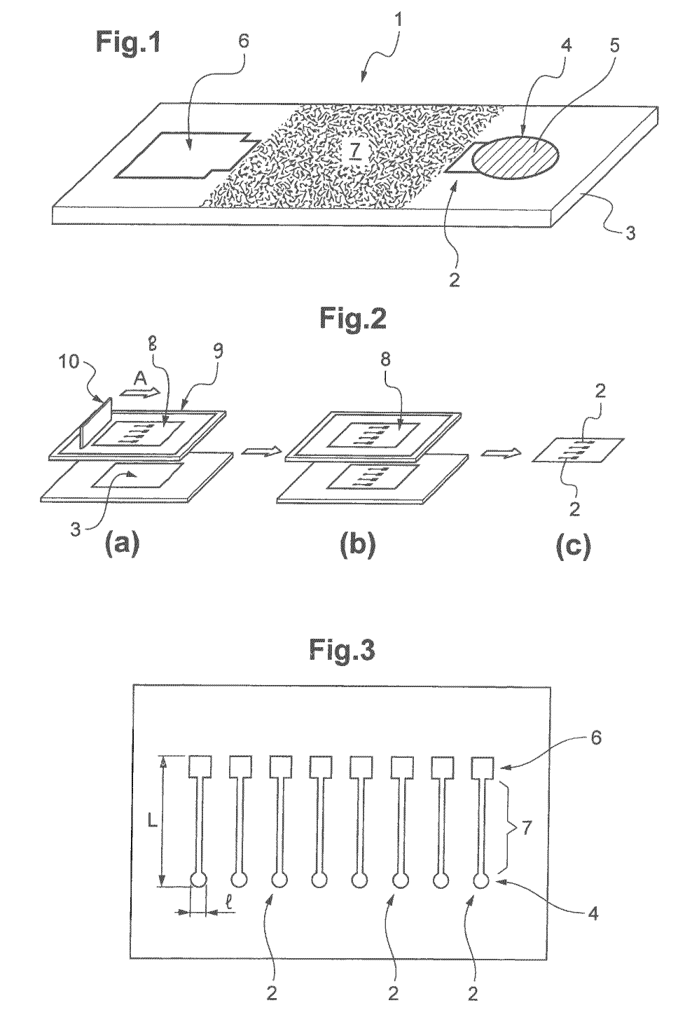

FIG. 1 is a schematic perspective view of a sensor according to the invention, in particular with its working electrode designed to be modified according to the invention,

FIG. 2 illustrates in perspective three steps (a), (b) and (c) for the preparation by screen printing of a series of working electrodes designed to be modified according to the invention,

FIG. 3 is a plan view of a series of working electrodes screen printed in this way and then further insulated,

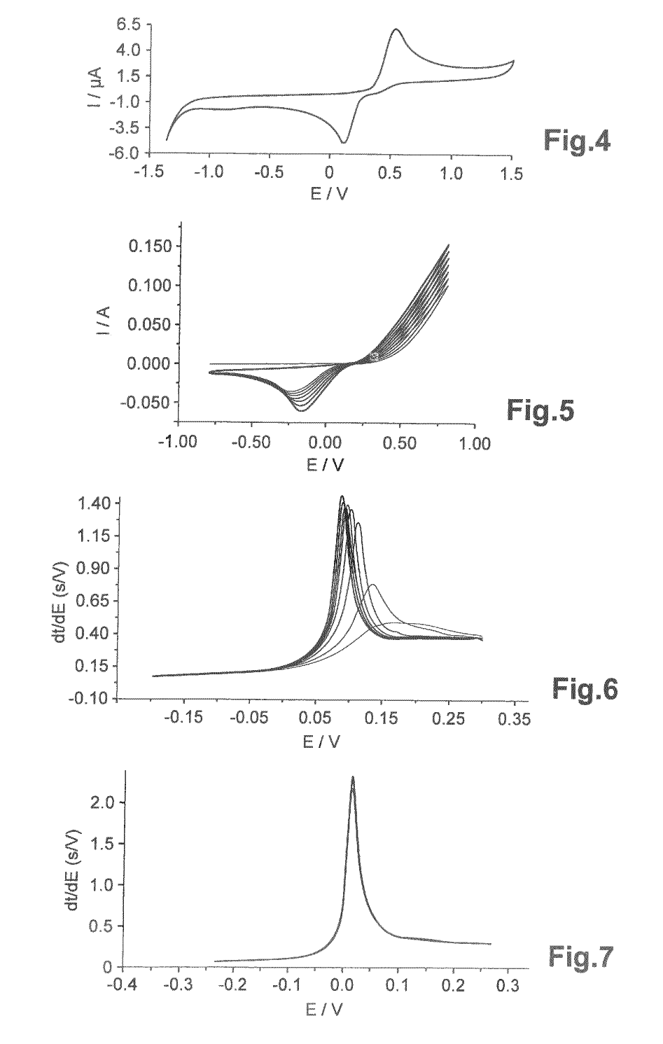

FIG. 4 is a cyclic voltammogram I (μA)=f [E(V)] obtained in a phosphate buffer solution (pH 7.2) containing pyrocatechol and ethylene diamine, during step a) of the invention,

FIG. 5 is a cyclic voltammogram I (μA)=f [E(V)] obtained in this phosphate buffer solution containing pyrocatechol and ethylene diamine, during step a) depending on the number of scans,

FIG. 6 is a graph of potentiometry in terms of the dt/dE (s/V)=f [E(V)] derivative, obtained during step c) of the invention in relation to conditioning with a view to detecting copper in an acetate buffer solution containing copper, following steps a) and b),

FIG. 7 is a graph of potentiometry in terms of the dt/dE (s/V)=f [E(V)] derivative, obtained during step c) of the invention in this acetate buffer solution containing copper, demonstrating the reproducibility of the signal (20 repetitions) for the detection of copper,

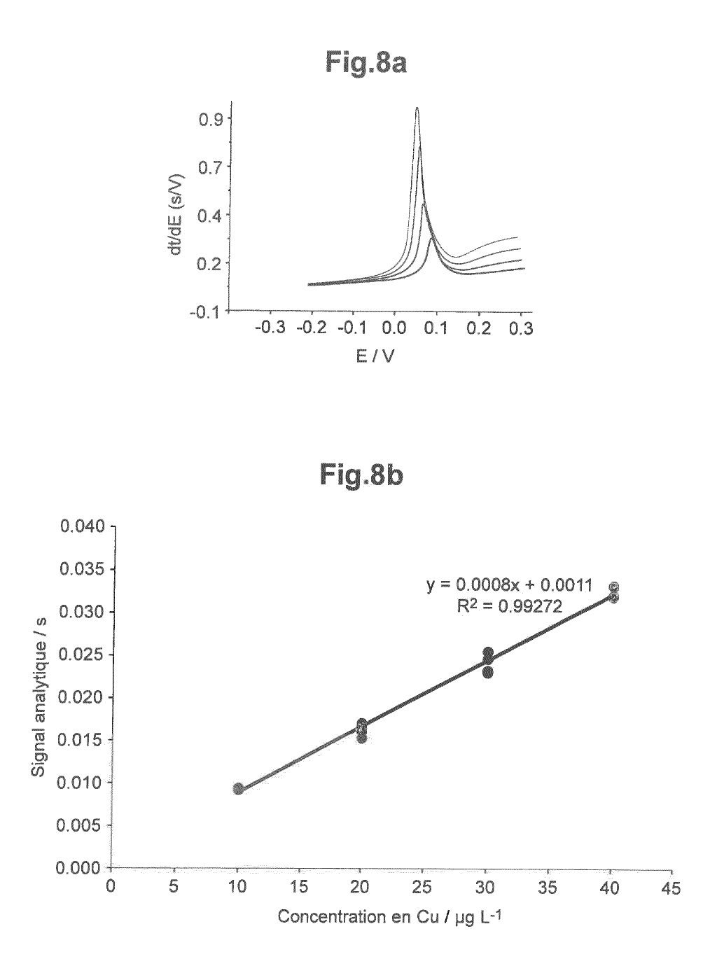

FIGS. 8a and 8b are respectively, for the measurement of copper, a graph of potentiometry dt/dE (s/V)=f [E(V)] and a calibration curve obtained for the peak of this graph, carried out in an acetate buffer with the addition of ethylene diamine in step a), demonstrating the evolution of the signal (s) as a function of copper concentrations of 10, 20, 30 and 40 μg/L,

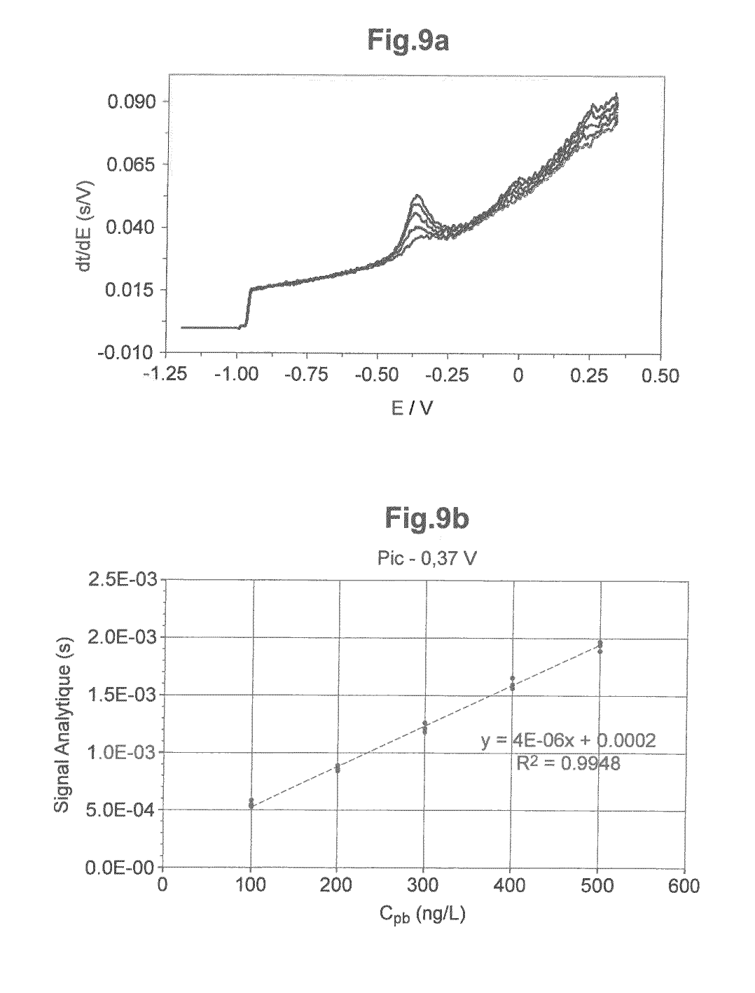

FIGS. 9a and 9b are respectively, for the measurement of lead, a graph of potentiometry dt/dE (s/V)=f [E(V)] and a calibration curve obtained for the peak of this graph, carried out in an acetate buffer without the addition of ethylene diamine in step a), demonstrating the evolution of the signal (in seconds) as a function of five lead concentrations of 100, 200, 300, 400 and 500 ng/L, and

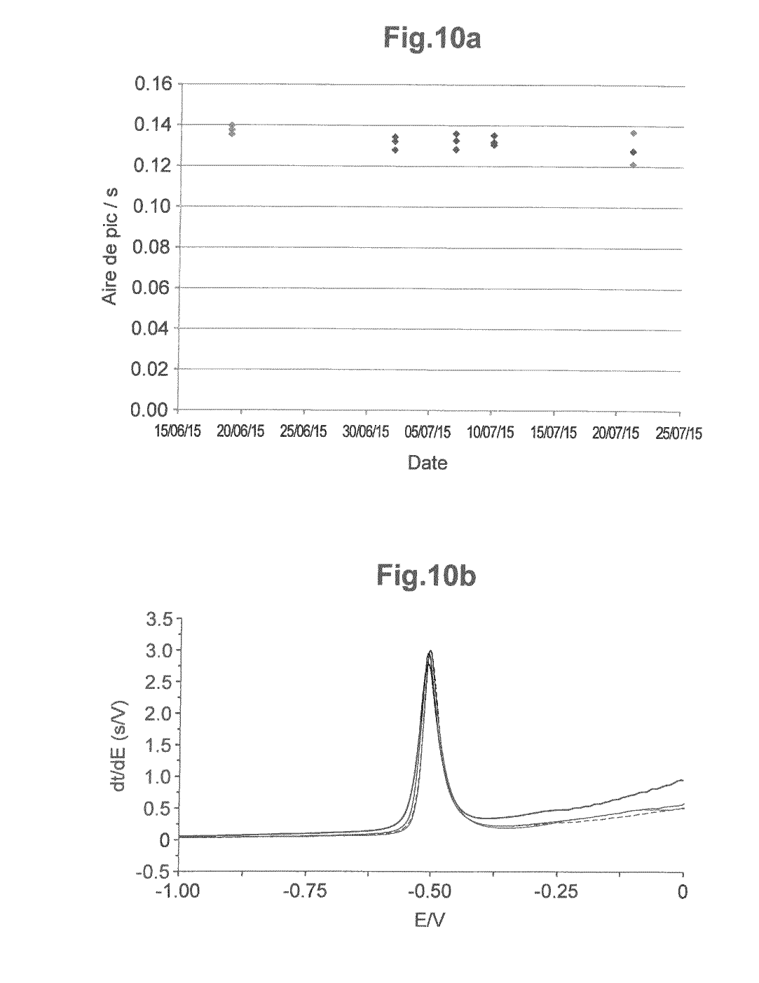

FIGS. 10a and 10b illustrate the results obtained in terms of the stability of working electrodes according to the invention following high-frequency measurements for one month, with FIG. 10a showing the analytical signals obtained (peak area/s) for a lead concentration of 40 μg/L and FIG. 10b showing the corresponding graph dt/dE (s/V)=f [E(V)].

The sensor 1 according to the invention illustrated in FIG. 1 during manufacture essentially comprises a working electrode 2 based on a carbon material (e.g. carbon in the form of graphite or carbon black) which is deposited by screen printing on an insulating support 3 (for example made of high-impact polystyrene) and which comprises a surface 4 which is designed to receive the polymeric film 5 according to the invention based on a poly(catechol) and separated from an electrical contact zone 6 by an electrically insulating layer 7 (for example made of high-impact polystyrene melted in mesitylene and deposited manually, or an insulating screen printing ink deposited by screen printing).

FIGS. 2(a) to 2(c) illustrate a method for the production of such working electrodes 2 by screen printing, with the known use of a stencil 8 forming a mask which has been interposed between the conductive ink 9 and the support 3 and which is defined by a mesh number (for example 200 mesh for the electrodes 2 manufactured according to the invention) or number of threads per inch (2.54 cm) corresponding to the mesh and consequently to the precision of the patterns to be obtained. A photosensitive resin was deposited in the centre of the stencil 8, and then a template of the working electrodes 2 to be made was arranged on this resin. The assembly was subjected to ultraviolet radiation in an imagesetter, and the resin crosslinked at the sites exposed to this radiation by becoming fixed to the stencil 8, in contrast to the areas masked by the template. The excess resin was removed with water and after air-drying for 48 hours, the patterns of the electrodes 2 were obtained, the stencil 8 forming a mask which was then ready for use.

As illustrated in FIG. 2(a), the insulating support 3 was arranged under the stencil 8 in such a way as to position it under the patterns of the electrodes 2. The conductive ink 9 (for example carbon graphite) was distributed at the top of the template and then spread in the direction of arrow A with a squeegee 10, preferably in two passes. Several working electrodes 2 were therefore made simultaneously and in a very short period of time.

Once screen printed, these electrodes 2 were air-dried for one hour, then dried in an oven at 60° C. for one hour, and the working electrodes 2 were obtained, as can be seen in FIGS. 2(b) and 2(c).

Then, and as illustrated in FIG. 3, these electrodes 2 were electrically insulated between their surface 4 and the electrical contact area 6 by the deposition of the insulating layer 7 which was applied manually using a spatula. Finally, the insulating layers 7 of the electrodes 2 were dried. In the tests carried out by the Applicant, the working surface obtained had a disc shape with a surface area equal to 9.6 mm² for each of the eight electrodes 2 (this working surface is represented by hatching in FIG. 1), which each had a width I of 1 cm and a length L of 5 cm in the example of FIG. 3.

Examples of Modifying the Working Electrodes 2 Using the Method According to the Invention:

A potentiostat-galvanostat marketed by METROHM under the name of pAutolab III was used, comprising, in connection with GPES software, a first part designed to switch on the system and to manage the data, a second part designed to control the stirring and the arrival of the stream of nitrogen and a third part containing the electrochemical device.

The tests were carried out in a voltammetry cell, with the electrochemical device which comprised three electrodes:
a screen-printed working electrode,
a graphite carbon counter electrode, and
a reference electrode (AgAgCl/KCl at 3 mol/L) fixing the potential.

In a known manner, the cyclic voltammetry that was used for step a) of the method of the invention (i.e. the deposition of the film 5 on each working electrode 2 by electropolymerisation) is based on the measurement of a current which is generated by a linear scan with a potential between the defined potentials and which results from the oxidation and reduction of the elements present in the solution. Each cycle is represented by a trace of the current recorded as a function of the potential applied. When scanning takes place from negative to positive potentials, the signal obtained corresponds to the oxidation of these elements (in the opposite case, the potential obtained is that of the reduction thereof).

FIGS. 4 and 5 show cyclic voltammograms obtained for this step a) according to the invention.

Also in a known manner, the stripping chronopotentiometry that was used for the steps b) of conditioning and c) of stabilising the method of the invention comprises:
electrodeposition on each electrode 2 by means of a sufficiently reducing potential applied for 60 seconds with stirring (in this way the metallic analytes are reduced and pre-concentrated), and
stripping by means of an oxidation current applied to re-oxidise the reduced metals, with the variation of the potential recorded as a function of time (the software actually calculates the inverse derivative of time with respect to the dt/dE potential, expressed in s/V as a function of the potential E, expressed in V).

FIGS. 6 and 7 show dt/dE=f(E) curves obtained during this step c).

1) Step a) of Electrodeposition According to the Invention:

To implement this step a), 7 to 50 successive scans were carried out with cyclic voltammetry from −0.8 V to +0.8 V at a speed of 200 mV/s without stirring, in a degassed phosphate buffer solution (with a pH ranging from 7 to 7.5, in particular including pH=7 and pH=7.2) containing pyrocatechol at a concentration of 10 mmol/L with or without ethylene diamine (where appropriate, used at two concentrations of 10 mmol/L and 12 mmol/L). The pyrocatechol was thus electropolymerised on the working surface of each carbon-based electrode 2.

For the solutions of pyrocatechol alone, the number of scans was 7 or 50, whereas for the solutions of pyrocatechol+ethylene diamine, the number of scans tested was 7, 10, 20, 30 and 50.

More specifically, the electropolymerisation of pyrocatechol was tested in particular by carrying out the following comparative tests:
pyrocatechol at 10 mmol/L in a soda solution (pH 12) at 0.1 mol/L,
pyrocatechol at 10 mmol/L in a phosphate buffer solution (pH ranging from 7 to 7.5) at 0.1 mol/L (test according to the invention),
pyrocatechol at 10 mmol/L in an acetate buffer solution (pH 4.6) at 0.1 mol/L,
pyrocatechol at 10 mmol/L+ethylene diamine at 10 mmol/L in a phosphate solution (pH ranging from 7 to 7.5) at 0.1 mol/L (another test according to the invention), and
pyrocatechol at 10 mmol/L+ethylene diamine at 12 mmol/L in a phosphate solution (pH ranging from 7 to 7.5) at 0.1 mol/L (another test according to the invention).

Furthermore, speeds of 200 mV/s, 300 mV/s, 400 mV/s and 500 mV/s were tested during these scans.

FIG. 4 illustrates the result obtained during the electropolymerisation of pyrocatechol at 10 mmol/L+ethylene diamine at 10 mmol/L in a 0.1 mol/L degassed phosphate solution (pH=7.2) from −0.8 V to +0.8 V at a speed of 200 mV/s without stirring. It has been verified that the anodic and cathodic peaks obtained in FIG. 4 correspond to the conversion of pyrocatechol into o-benzoquinone and vice versa, with the exchange of two electrons.

And FIG. 5 shows that, when these scans are carried out in this 0.1 mol/L degassed phosphate solution (pH=7.2) from −0.8 V to +0.8 V at 200 mV/s with mmol/L of pyrocatechol and 10 mmol/L of ethylene diamine, a decrease in the height of the reduction peak is observed with the increase in the number of scans. This evolution provides evidence of the formation of the polymeric film 5 according to the invention on the surface of each carbon electrode 2, this film 5 being even thicker when the number of scans is higher.

Therefore, the addition of ethylene diamine to the phosphate buffer solution with a substantially neutral pH containing the pyrocatechol is a preferred embodiment of the invention.

2) Step b) of Conditioning the Film According to the Invention:

To implement step b), each modified electrode was subjected, after step a), to stripping chronopotentiometry in a solution of NaOH at 0.1 mol/L for a period ranging from 60 seconds to 600 seconds.

Different solutions were tested:
in soda at 0.1 mol/L (test according to the invention),
in a phosphate solution at 0.1 mol/L (blank test),
in an acetate solution at 0.1 mol/L (blank test).

For each time period and solution tested, we used:
10 cycles of stripping chronopotentiometry comprising 1 degassing step of 60 seconds at +0.3 V, then conditioning for 60 seconds at +0.3 V, then a depositing step at −0.8 V for 60 seconds followed by a stripping step (applying an oxidising current of 10 μA);
7 scans with cyclic voltammetry from −0.4 V to +1 V at a speed of 100 mV/s; and
1 cycle of stripping chronopotentiometry comprising 1 degassing step of 600 seconds at +0 V, then a conditioning step of 600 seconds carried out at +0.3 V, then a depositing step at −1.2 V for 60 seconds followed by a stripping step (applying an oxidising current of 10 μA).

The 10 cycles of stripping chronopotentiometry for 60 seconds in the soda solution have been identified as the best conditioning, in accordance with step b) according to the present invention.

In fact, these measurements have demonstrated that the electrodes modified after step a) of the invention but which were not subjected to conditioning in soda solution do not generate a signal in stripping chronopotentiometry, according to the dt/dE=f(E) graphs obtained for conditioning in the aforementioned “blank” solutions of phosphate at 0.1 mol/L and acetate at 0.1 mol/L.

The Applicant has verified that this step b) makes it possible to make the film electroactive in a range from −1 V to +1 V, therefore advantageously making it possible to detect metallic elements with standard oxidation-reduction potentials within this range.

It seems that conditioning in soda makes it possible to functionalise the film by making it electroactive and thus facilitate the complexation of the elements to be detected, such as copper and lead.

3) Step of Drying the Film According to the Invention:

The activated film was dried after step b) using different drying times in ambient air:

0 hours, 3 hours, 4 hours and 24 hours of drying (24 hours being the best embodiment), it having been specified, however, that a lack of drying (i.e. 0 hours) did not penalise the activation or the other properties of each film obtained.

4) Stage c) of Stabilising the Film According to the Invention:

Each working electrode coated with the film obtained after step b) and having been dried for 24 hours was conditioned again by stripping chronopotentiometry in an acetate buffer solution at 0.1 mol/L (pH=4.6) containing the metallic element to be detected, i.e. containing 100 ng/L to 40 μg/L of copper and/or lead. More specifically, 30 repetitions or cycles were carried out—with prior use in step a) of two phosphate buffer solutions, with ethylene diamine respectively added and not added, it having been specified that the stability of the film was obtained in these two cases after 25 repetitions in step c).

The Applicant has verified that these two buffer solutions with and without ethylene diamine in step a) ensure stabilisation of the film by eliminating the undesirable monomers in step c) which were possibly deposited on the working electrode, except for the fact that the solution with ethylene diamine makes it possible to obtain an even better stability of the film and this is achieved more quickly. This therefore confirms the fact that the addition of ethylene diamine in step a) is a preferred embodiment of the invention.

FIG. 6 shows the stripping chronopotentiometry curves obtained during this step c) of conditioning in the acetate buffer solution at 0.1 mol/L (pH 4.6) containing 40 μg/L of copper.

FIG. 7 shows the stripping chronopotentiometry curve obtained during this step c) of conditioning, and this FIG. 7 provides evidence of the excellent repeatability of the signal since a coefficient of variation of less than 5% was obtained over 20 repetitions in this acetate buffer solution at 0.1 mol/L (pH 4.6) containing 40 μg/L of copper. In fact, the repeatability obtained is considered to be satisfactory when the coefficient of variation is less than 10% over a multitude of repetitions.

Validation Test Report for Sensors According to the Invention Incorporating Working Electrodes Modified in this Way for the Detection of Copper and Lead:

1) Detection of Copper:

As illustrated in FIGS. 8a and 8b, a calibration curve was obtained in four acetate buffer solutions with a pH of 4.6 containing four copper concentrations per stripping chronopotentiometry respectively (calibration with a peak at +0.06 V, see the four dt/dE=f [E] curves of FIG. 8a), after having previously subjected this solution to degassing for 10 minutes. The analytical conditions were as follows:
purging for 60 seconds at +0.3 V,
conditioning for 60 seconds with stirring at −0.3 V,
depositing for 60 seconds at a deposition potential Ed of −1.2 V with stirring,
equilibrating for 10 seconds at −1.2 V without stirring, and
stripping current of 10 μA.

For this test, we used a film obtained:
by step a) in a phosphate buffer solution at 0.1 mol/L (pH 7.2) containing 10 mmol/L of pyrocatechol and 10 mmol/L of ethylene diamine by cyclic voltammetry from −0.8 V to +0.8 V at 200 mV/s without stirring,
by step b) implemented for 60 seconds in a soda solution at 0.1 mol/L,
by drying in ambient air for 24 hours, then
by step c) in this acetate buffer solution at 0.1 mol/L.

As can be seen in FIG. 8b, four measurement points were respectively carried out at 10, 20, 30 and 40 μg/L of copper, each repeated five times. FIG. 8b shows good linearity obtained in the concentration range from 10 to 40 μg/L, with good sensitivity of the sensor given the signals obtained after 60 seconds of analysis.

A limit of detection (LOD) for copper in solution equal to 1.3 μg/L was obtained by a probability calculation, with an operating period of several weeks for the sensors incorporating the working electrodes modified in this way.

2) Detection of Lead:

As illustrated in FIGS. 9a and 9b, a calibration curve was obtained in the aforementioned acetate buffer solution at pH 4.6 by stripping chronopotentiometry, after having subjected this solution to degassing for 10 minutes and using the following analytical conditions:
Deposition potential Ed=−1.2V for 60 seconds,
equilibrium time 10 seconds,
limit potential E_{I}=+0.3 V,
impressed current 10 μA.

For this test, we used a film obtained:
by step a) in a phosphate buffer solution at 0.1 mol/L (pH 7.2) containing 10 mmol/L of pyrocatechol by cyclic voltammetry from −0.8 V to +0.8 V at 200 mV/s without stirring,
by step b) for 60 seconds in soda at 0.1 mol/L,
by drying in ambient air for 24 hours, then
by step c) in the acetate solution at 0.1 mol/L.

As can be seen in FIG. 9a, calibration was obtained with a peak of −0.37 V for five dt/dE=f(E) curves corresponding respectively to five concentrations of lead in the acetate solution. For these five dt/dE=f(E) curves, a distinct signal was obtained at a lead concentration of 100 ng/L (see FIG. 9b), with an increase in the peak in proportion to the concentration of lead, which provides evidence of the ability to detect and quantify lead in solution (just like copper) with good sensitivity.

As can be seen in FIG. 9b, five measurement points were respectively carried out at 100 ng/L, 200 ng/L, 300 ng/L, 400 ng/L and 500 ng/L, each repeated five times. FIG. 9b shows good linearity obtained in the concentration range from 100 to 500 ng/L, with good sensitivity of the sensor given the signals obtained after 60 seconds of analysis.

A limit of detection (LOD) for lead in solution equal to 63 ng/L and a limit of quantification (LOQ for short, with LOQ=[b+(10·σ_{b})]/a) of 92 ng/L were obtained by means of probability calculations for these tests, with an operating period of the sensors incorporating the working electrodes modified in this way being several weeks.

In order to verify the stability of the working electrodes 2 obtained by the method of the invention presented above for the detection of trace lead, a series of high-frequency measurements were carried out for one month, changing the solution regularly in order to maintain identical experimental conditions.

FIG. 10a shows the analytical signals obtained after each change of solution for a Pb concentration of 40 μg/L. It appears that each working electrode 2 was in continuous operation at the rate of one measurement every 30 minutes, according to the same procedure of the invention as described above.

FIG. 10b shows that, throughout this one-month period, the signals obtained with the aid of each working electrode 2 remained stable.

1. A method for the modification of a carbon material surface of a working electrode for a sensor which is able to detect at least one dissolved trace metallic element in a liquid, the method comprising a step a) of depositing a polymeric film based on a poly(catechol) on said surface by electropolymerisation of the pyrocatechol in a first environment with a pH of between 6 and 8,
the method further comprising a step b) of conditioning said working electrode comprising a succession of reduction-oxidation cycles in a second environment with a pH equal to or greater than 10,
wherein the method further comprises, after step b), a step c) of stabilising said working electrode comprising a repetition of other reduction-oxidation cycles in a third environment which has a pH of between 4 and 5 and which comprises said at least one metallic element to be detected.

2. The method according to claim 1, wherein step b) is implemented in a soda solution which is free from said at least one metallic element and which preferably has a pH of between 11 and 13.5.

3. The method according to claim 1, wherein step b) is implemented by stripping chronopotentiometry, carrying out between 5 and 15 of said reduction-oxidation cycles.

4. The method according to claim 3, step b) is implemented for each of said cycles with the application of a reduction and then oxidation potential for a period of between 30 seconds and 90 seconds.

5. The method according to claim 1, wherein said third environment is an acetate buffer solution preferably at 0.1 mol/L which comprises copper and/or lead as said at least one metallic element at a concentration of between 50 ng/L and 100 μg/L.

6. The method according to claim 1, wherein step c) is implemented by stripping chronopotentiometry, carrying out at least 25 of said other cycles.

7. The method according to claim 1, wherein step a) is implemented in an environment comprising a phosphate buffer solution at 0.1 mol/L which has a pH of between 6.5 and 7.5 and which comprises said pyrocatechol at a concentration of between 5 mmol/L and 15 mmol/L.

8. The method according to claim 7, wherein step a) is implemented by cyclic voltammetry, with a plurality of scans from −0.8 V to +0.8 V carried out at a speed of at least 200 mV/s.

9. The method according to claim 1, wherein step a) further comprises an initial addition of ethylene diamine to said first environment, at a concentration of between 5 mmol/L and 15 mmol/L.

10. A working electrode for a sensor which is able to detect at least one dissolved trace metallic element in a liquid, the working electrode having a surface based on a carbon material coated with a polymeric film based on a poly(catechol), said film defining a working surface of the working electrode, said working surface comprising the product of reduction-oxidation reactions by stripping chronopotentiometry of said poly(catechol) with a soda solution which functionalises it, the working electrode being electroactive in a field of electroactivity, measured by cyclic voltammetry, ranging from −1 V to +1 V while being able to detect copper and/or lead as said at least one metallic element
wherein said working surface further comprises the product of reduction-oxidation reactions by stripping chronopotentiometry of said poly(catechol) with an acetate buffer solution which comprises copper and/or lead as said at least one metallic element at a concentration of between 50 ng/L and 100 μg/L, the copper and/or lead having reacted with said film.

11. A sensor which is able to detect at least one dissolved trace metallic element in a liquid, the sensor comprising a working electrode, a counter electrode and a reference electrode, wherein said working electrode is as defined in claim 10, the sensor being able to detect said metallic elements which have a standard oxidation-reduction potential of between −1 V and +1 V and which include copper and lead.

12. The sensor according to claim 11, wherein the sensor has a sensitivity, expressed as the limit of detection of said at least one metallic element, which is less than 1.5 μg/L and is less than 70 ng/L for lead.

13. Use of a sensor according to claim 11 to detect and possibly quantify in situ, for example in a monitoring station, said at least one metallic element in solution in industrial waters or baths, before and/or after their treatment, which are selected, for example, from surface waters, groundwater, industrial discharge, water from sewage treatment plants, drainage and runoff water, water from polluted and unpolluted soils and leachate from landfills.
